(19) United States
(12) Patent Application Publication
(10) Pub. No.: US 2010/0324561 A1
(43) Pub. Date: Dec. 23, 2010

(54) METHOD AND DEVICE FOR CARRYING OUT A PERCRESTALE SINUS LIFT

(75) Inventors: Georg Watzek, Wien (AT); Ewald Unger, Wien (AT)

Correspondence Address: JOYCE VON NATZMER;PEQUIGNOT + MYERS LLC, 200 Madison Avenue, Suite 1901, New York, NY, 10016, US

(73) Assignee: MEDIZINISCHE UNIVERSITAET WIEN, Wien (AT)

(21) Appl. No.: 12/742,872

(22) PCT Filed: Nov. 14, 2008
(86) PCT No.: PCT/AT2008/000414

§ 371 Date: Aug. 18, 2010

(30) Foreign Application Priority Data
Nov. 15, 2007 (AT) A 1858/2007

(51) Int. Cl. A61B 17/16; A61B 17/58

(52) U.S. Cl. 606/80; 606/92

(57) ABSTRACT

The invention relates to a method for carrying out a percrestale sinus lift, wherein a bore channel is created with the aid of a boring, milling and/or hammering method, the Schneider membrane is lifted from the jaw bone using a flowable medium that is supplied via the opened bore channel, bone replacement material is introduced, a supply line is introduced into the bore channel prior to introducing the flowable liquid and is sealingly fixed in a detachable manner to the bore channel or onto the edge thereof and the flowable medium in introduced through the supply line. The invention also relates to a surgical instrument (1) for introducing a flowable medium for lifting the Schneider membrane from the jaw bone whilst carrying out a percrestale sinus lift. Said instrument comprises a supply line that can be introduced into the bore channel and that has at least one sealing element for sealing and detachably fixing the supply line to the bore channel or to the edge thereof, a displaceable sleeve (4) on the supply line is guided in the axial direction in relation to the supply line and the sealing element (7) is arranged between a front surface of the sleeve (4) and a surface, preferably a ring surface (6), protruding in the radial direction from the supply line, and can be deformed by axially pressing the sleeve (4) in the radial direction and can be pressed counter to the inner wall of the bore channel (3).

The invention relates to a method for performing a percrestal sinus lift, in which a bore channel is created with the aid of drilling, milling and/or hammering methods, the Schneiderian membrane is lifted from the jaw bone using a free-flowing medium supplied through the opened-up bore channel and bone-replacement material is introduced. Furthermore, the invention relates to a surgical instrument for introducing a free-flowing medium for lifting the Schneiderian membrane from the jaw bone when performing a percrestal sinus lift and an instrument set for performing a percrestal sinus lift.

In the case of existing edentia, there is usually little bone available for anchoring an endosseous tooth implant due to pneumatization of the maxillary sinus. This lack of bone can be rectified by so-called sinus lift surgery, in which bone-replacement material is introduced between bones and the Schneiderian membrane. This surgical technique requires intact detachment of the Schneiderian membrane from the maxillary sinus in order to create a cavity for the bone material to be introduced. Herein, the bore hole for the implant is advanced to the direct vicinity of the Schneiderian membrane and, if need be, the remaining osseous lamina is pierced by a further instrument, such as an osteotome, whereupon the Schneiderian membrane can be detached from the base of the maxillary sinus for example with the aid of a balloon catheter. However, this method harbors a significant risk of destroying the very sensitive Schneiderian membrane, and so it appears desirable to carry out the lifting of the membrane in a more gentle fashion and whilst avoiding tension spikes in the membrane.

EP 1 362 561 A2 describes a device for detaching the maxillary sinus membrane. Here, the preparation device for a sinus base elevation has a pump with open-loop or closed-loop control, which supplies a preparation medium through a tube connection up to an access opening to be brought about in the jaw bone, wherein the preparation device has an attachment means allowing an attachment of the tube to the jaw bone by generating a negative pressure, like in a suction cup. The jet of preparation medium passing through the access opening is directed at the maxillary sinus membrane in order to detach the latter from the jaw bone in a gentle fashion. Herein, the access opening to the maxillary sinus membrane is created by conventional instruments. A disadvantage of this device and the corresponding method is that, inter alia, there is significant technical complexity in fastening the attachment means on the jaw bone by means of negative pressure and introducing the preparation medium, with it not always being possible to ensure a secure hold of the attachment means on the jaw bone. It should be considered particularly disadvantageous that if the preparation medium is not simultaneously used as the bone-replacement material for the subsequent sinus elevation, said preparation medium comes into significant contact with the cut surfaces in the jaw bone and can penetrate the spongy structure of the bone, which is disadvantageous, particularly when an X-ray contrast agent is also used in the preparation medium.

The object of the invention now is to develop a method for performing a percrestal sinus lift, in which the Schneiderian membrane can be gently lifted from the jaw bone using a free-flowing medium supplied via the opened-up alveolar canal or the defined bore channel, wherein the aforementioned disadvantages should be avoided.

In order to achieve this object, the method according to the invention for performing a percrestal sinus lift essentially consists of a supply line being inserted into the bore channel prior to the supply of the free-flowing medium and being detachably affixed in a sealing fashion in the bore channel or to the edge thereof, and the free-flowing medium being introduced through the supply line. As a result of this, a supply line can be affixed with little technical complexity, with it being ensured at the same time that the cut surfaces on the jaw bone do not come into contact with the free-flowing medium and so the latter cannot penetrate the spongy bone. Here, the free-flowing medium for lifting the Schneiderian membrane from the jaw bone can directly be the bone-replacement material or a medium differing from this material, wherein if a medium differing from the bone-replacement material is used, it has to be removed from the space between the jaw bone and the Schneiderian membrane after the lifting procedure.

Here, the method can preferably be developed such that the supply line is affixed in the bore channel or to the edge thereof with the aid of a flexible annular seal, wherein the annular seal is preferably deformed or expanded in the radial direction and pressed against the inner wall of the bore channel. This allows the supply line to be affixed in a gentle fashion whilst also avoiding the introduction of force spikes in the jaw bone with the spongy structure of the jaw bone being screened at the same time from the free-flowing material to be introduced in an optimal fashion. In particular, as a result of the supply line only reaching its effective diameter after being inserted into the bore channel, a caving-in of the possibly very thin maxillary bone structure can be avoided when inserting the supply line.

Herein, the method can preferably be developed such that an annular seal designed as a tubular seal or balloon seal is pressed against the inner wall of the bore channel by the application of pressure, which further decreases the tensional stresses in the bore channel.

Herein, the method is advantageously performed such that the supply line is affixed in the bore channel in a sealing fashion at its front end in the insertion direction. This allows, firstly, the entire cut region of the jaw bone to be protected where possible from undesired perfusion by the free-flowing medium and, secondly, the risk of damaging the Schneiderian membrane by regions of the supply line protruding distally from the annular seal to be minimized.

A gel is advantageously used as free-flowing medium for lifting the Schneiderian membrane from the base of the maxillary sinus. In this context, the use of a gel offers the advantage of the gel firstly ensuring an even load distribution in the Schneiderian membrane and thus avoiding as far as possible the occurrence of undesired tension spikes in the Schneiderian membrane when lifting it from the jaw bone, whilst secondly placing relatively tight restrictions on the region in which the Schneiderian membrane is to be lifted from the base of the maxillary sinus compared to a less viscous medium. This affords optimal control of the success of the lift on the basis of the volume of the introduced free-flowing medium.

As already mentioned above, the method according to the invention can preferably be developed in that a highly viscous bone-replacement material is selected as free-flowing medium, and so rinsing of the material used to lift the Schneiderian membrane from the base of the maxillary sinus can be dispensed with and the implant can, if need be, be placed directly after the sinus lift.

The invention furthermore relates to a surgical instrument for introducing a free-flowing medium for lifting the Schneiderian membrane from the jaw bone when performing a percrestal sinus lift, which instrument is particularly suitable for performing the described method. Herein, the instrument has a supply line, which can be inserted into the bore channel and has at least one sealing element for affixing the supply line in the bore channel or to the edge thereof in a sealing and detachable fashion, and is characterized in that a sleeve that can be displaced relative to the supply line in the axial direction is guided along the supply line and in that the sealing element is arranged between an end face of the sleeve and a face, more particularly an annular face, projecting from the supply line in the radial direction, can be deformed by axial pressing of the sleeve in the radial direction, and is designed such that it can be pressed against the inner wall of the bore channel. This allows the surgical instrument to be brought into the desired position without straining the jaw bone and thereupon to be affixed in a detachable fashion in the bore channel or to the edge thereof by displacing the sleeve, which leads to a radial expansion of the annular seal.

Advantageously, the surgical instrument is preferably developed to the effect that the sealing element has at least one flexible annular seal, wherein the annular seal is advantageously designed such that it can be deformed in the radial direction and can be pressed against the inner wall of the bore channel. Such a sealing element can be brought to rest against the cut surface in the jaw bone in a particularly gentle fashion and so there can be optimal screening of the spongy structure of the jaw bone whilst at the same time avoiding the introduction of force spikes into the bone.

Advantageously, the surgical instrument is preferably designed such that the annular face has securing means to prevent the sealing means from slipping off. Such securing means can be, for example, an encircling elevation directed in the proximal direction. In this context, it is also feasible for the annular face to be designed such that it is angled toward the inside in the insertion direction and so the sealing means is held radially inwardly.

Herein, the surgical instrument is preferably developed such that the end face of the sleeve facing the sealing element is designed as an angled face, more particularly a frustum-shaped face, facing away from the supply line, as a result of which even on the application of only a small force by the operator in the axial direction a sufficiently large radial force is applied from the sealing element to the surrounding jaw bone, with it simultaneously being ensured that the radial extent of the annular seal remains within justifiable boundaries and hence damage to the jaw bone is avoided.

Advantageously, the surgical instrument can be developed to the effect that the sleeve interacts with a tensioning and/or fastening element for tensioning or fastening the sleeve in a position pressed against the sealing element and so the surgical instrument remains in the desired position without further action once it has been brought into the desired position. According to a preferred embodiment of the invention, the tensioning and/or fastening element can be formed by a screw that can be screwed against the sleeve in the axial direction, allowing the surgical instrument to be affixed in the bore channel or to the edge thereof in a particularly simple fashion. According to a further preferred embodiment of the present invention, provision is made for the tensioning or fastening element to be formed by a slider acting like a wedge, the face of the slider angled relative to the axis of the sleeve interacting with a ramp face of the sleeve during the lateral insertion in order to press the sleeve against the sealing element in the axial direction. Such an embodiment of the tensioning and/or fastening element allows the surgical instrument to be anchored in the bore channel using only one hand. An eccentric lever can be provided as a further possible embodiment of the tensioning and/or fastening element, which lever is fastened to part of the body of the surgical instrument and moves the sleeve distally by being tilted.

According to a further preferred embodiment of the present invention, provision is made for the sealing element to be arranged at the front end of the supply line in the insertion direction, as a result of which the surgical instrument can be anchored in the direct vicinity of the Schneiderian membrane. The arrangement of the sealing element at the front end of the supply line in the insertion direction therefore allows firstly the jaw bone to be protected over its entire cut surface, while, at the same time, damage to the Schneiderian membrane by regions of the surgical instrument protruding too far beyond the annular seal in the distal direction is avoided.

In order to protect the Schneiderian membrane in an optimum fashion from perforation by the surgical instrument to be inserted, provision is made according to a preferred development of the invention for the supply line and/or the sleeve to have a stop for limiting the insertion depth into the bore channel. Herein, the stop for limiting the insertion depth into the bore channel can be arranged at an appropriate distance from the sealing element and/or can be defined by distance elements such as annular disks such that the surgical instrument can be inserted into the bore channel without risk until the stop on the surgical instrument interacts with the gum or an implantation rail, whereupon the surgical instrument is affixed in the bore channel or to the edge thereof.

Alternatively, provision can also be made for the annular seal to be designed as an inflatable tubular seal or balloon seal, wherein provision is made for a channel opening into a cavity of the seal for supplying a pressure medium such that the annular seal is inflated when pressure is supplied and can be pressed against the inner wall of the bore channel. This affords a particularly gentle seal in the sinus lift region for the introduction of the free-flowing medium.

The invention furthermore relates to an instrument set for performing a percrestal sinus lift. The instrument set comprises a drilling, milling and/or hammering instrument for creating an access to the Schneiderian membrane, an instrument according to the invention as described above for supplying a free-flowing medium via the bore channel in order to lift the Schneiderian membrane from the jaw bone and an instrument, preferably separate from the other instruments, for introducing a bone-replacement material. Such a set offers optimum preconditions for performing the described method by providing the instrument(s) for creating a bore channel, the surgical instrument for introducing a free-flowing medium for lifting the Schneiderian membrane from the jaw bone as per one of claims 8 to 17 and a further instrument, preferably separate from the other instruments, for introducing bone-replacement material.

There is a danger of the maxillary sinus membrane tearing if the drill pierces the bone and is not removed in good time, particularly during the introduction of the bore into the jaw bone and when the bore channel pierces the sinus.

Now, in order to prevent the Schneiderian membrane being damaged, the suggestion has already been made to use not only one drill, but a drill set in the surgical intervention, in which drill set a substantially semicircular drill body has at least some flat surfaces, wherein the flat part of the drill body can be shaped like a cup or the shell of a partial sphere and, if need be, can be kept stationary as a separate part compared to the drill body provided with a material-removing surface structure. As soon as the flat part of the drill body of such a drill comes into contact with the Schneiderian membrane, it should firstly be ensured that this part does not continue to rotate in order to avoid damage to the membrane and, secondly, that drilling with the remaining regions with a material-removing surface structure is still possible. Such drills are generally provided with apparatuses for supplying and discharging a cooling and/or rinsing liquid, with such drills and drill sets, firstly, being technically relatively complex because the drills have a multipart design in the case of only small dimensions and, secondly, it is unable to guarantee that these drills will not perforate the Schneiderian membrane if the piercing of the jaw bone is not recognized in time by the operator.

It is now an object of the invention to develop a drill set by means of which excessive penetration of the drill into the maxillary sinus and hence perforation of the Schneiderian membrane can be effectively avoided and hence the risk to the patient can be minimized.

In order to achieve this object, the subject matter of the invention furthermore relates to a drill set for use when performing a percrestal sinus lift, which has at least one drill of a first type, which is designed as a pilot drill, and at least a first drill of a second type, which has a non-cutting cylindrical body with a diameter not exceeding the drill diameter of the drill of the first type and a drill pin protruding from the distal end face of the cylindrical body by a defined length. Such a drill set offers the possibility of drilling in the jaw bone with the drill of the first type to just in front of the Schneiderian membrane, whereupon a drill of the second type can be used to bring about the actual penetration up to the Schneiderian membrane. The drill of the second type forms a stop on the non-cutting cylindrical body, which stop interacts with the base of the bore created by the drill of the first type, and so the drill pin is only able to advance by the defined length by means of which the drill pin protrudes from the cylindrical body. Known imaging methods allow the pilot bore to be drilled into, for example, a planned region 2 mm in front of the Schneiderian membrane such that, if the drill pin projecting from the cylindrical body of the first drill of the second type for example has a length that is 2 mm less than the display error of the imaging method and the evaluation system, perforation of the Schneiderian membrane can be precluded because even if the drill of the first type cannot be positioned accurately in the jaw bone due to the error, excessive penetration into the maxillary sinus can nevertheless be reliably precluded.

Advantageously, the drill set according to the invention is developed to the effect that at least a second drill of the second type has a drill pin protruding from the distal end face of the cylindrical body by a greater defined length than that length of the drill pin of the first drill of the second type. Such a drill set in turn allows a pilot bore as described above to be drilled and an approach to the through-hole to the Schneiderian membrane can be implemented by a plurality of drills of the second type, wherein the non-cutting cylindrical body of the drills of the second type in each case interacts with the base of the pilot bore and the advance of the bore is in each case brought about by the stepwise enlargement of the respective drill pins of the drills of the second type. Subsequently, the surgical instrument as per claims 8 to 17 can in turn be used for introducing the free-flowing medium for lifting the Schneiderian membrane from the jaw bone. Once the Schneiderian membrane has been lifted off, the pilot drill can expand the through-hole in the jaw bone to the actually desired diameter without the risk of damage to the Schneiderian membrane, whereupon, if need be by using a further instrument, the desired amount of bone-replacement material can be introduced into the sinus.

According to a preferred development of the drill set according to the invention, the drills of the first and the second type each have channels for supplying and/or discharging cooling and/or rinsing liquid, wherein the channels for supplying the cooling and/or rinsing liquid preferably open into the region of the drill pin. Cooling the surgical site is generally indispensible when making such channels in order to avoid the necrosing of the bone tissue due to the influence of heat, wherein, secondly, if the channels for supplying the cooling and/or rinsing liquid open out in the region of the drill pin, a swimming water screen develops in front of the drill pin when the jaw bone is pierced toward the Schneiderian membrane and this screen keeps the membrane away from the rotating drill pin.

Advantageously, the drill set according to the invention is developed to the effect that the drill pin of the at least one drill of the second type is designed as a gun drill with a semicircular drill tip, wherein this geometry with a semicircular drill tip has already proven its worth in the field of the invention. It allows both precise and minimally traumatic removal of the bone material and satisfactory removal of the cut material.

Advantageously, the drill set is developed to the effect that the drill pin of the at least one drill of the second type is designed as a diamond-finished drill tip. Diamond-finished drill tips particularly distinguish themselves in the field of the invention in that they allow efficient cutting of bone material, with soft tissue, such as the Schneiderian membrane in particular, being particularly spared.

In the following text, the invention will be explained in more detail on the basis of an exemplary embodiment schematically illustrated in the drawing. In the latter,

FIG. 1 shows a sectional illustration of a surgical instrument for introducing a freely-flowing medium for lifting the Schneiderian membrane from the jaw bone when performing a percrestal sinus lift according to the present invention,

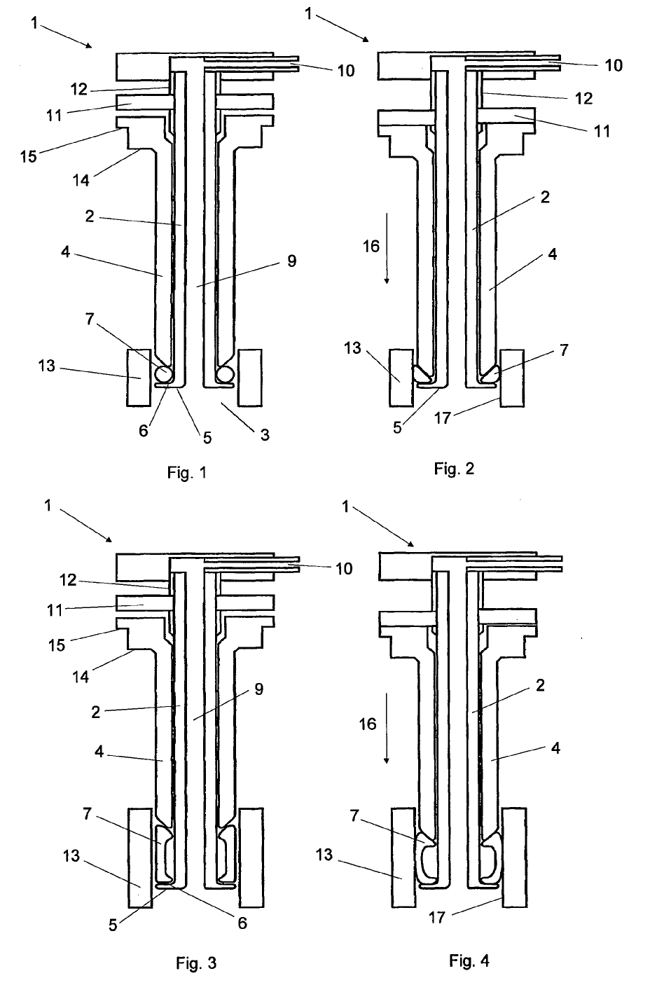

FIG. 2 shows an instrument corresponding to FIG. 1, in which the sealing ring for affixing the instrument was radially deformed in the bore channel,

FIGS. 3 and 4 show an analogous surgical instrument with an alternative embodiment of the sealing element,

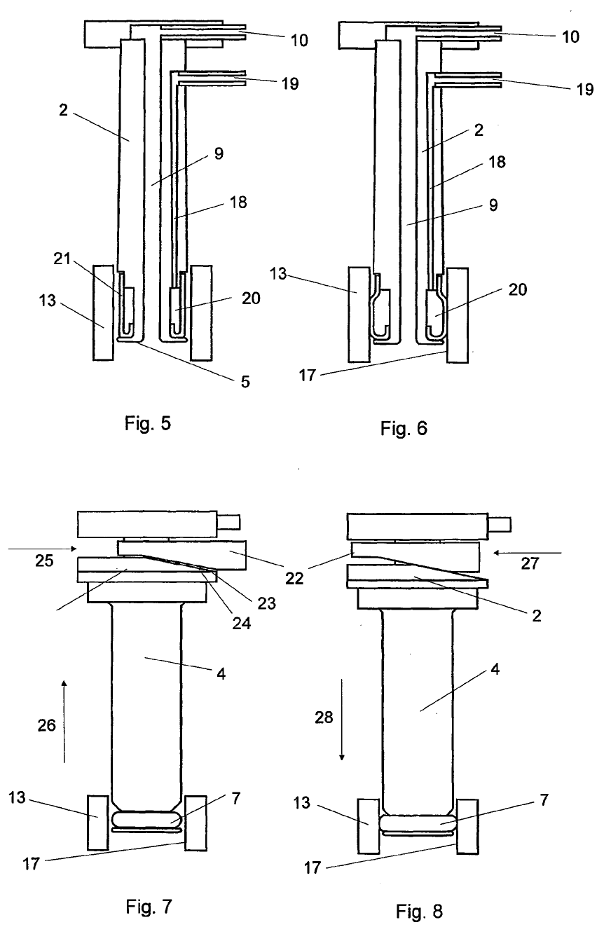

FIGS. 5 and 6 show a surgical instrument with a further alternative embodiment of the sealing element,

FIGS. 7 and 8 show a surgical instrument according to the invention, in a side view, with an alternative tensioning and/or fastening element,

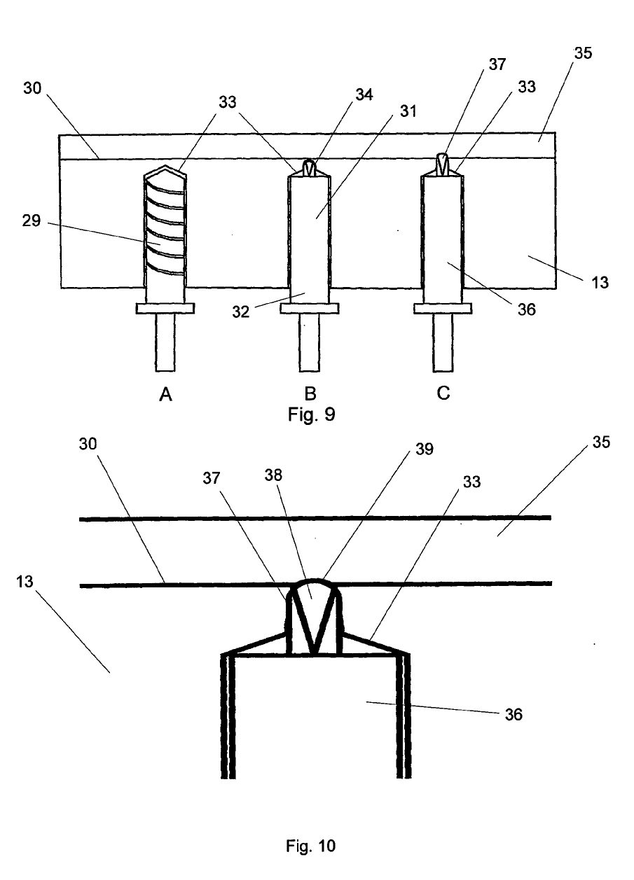

FIG. 9 shows a sectional illustration of a drilling sequence when using the drill set according to the invention,

FIG. 10 shows a detailed view of FIG. 9C,

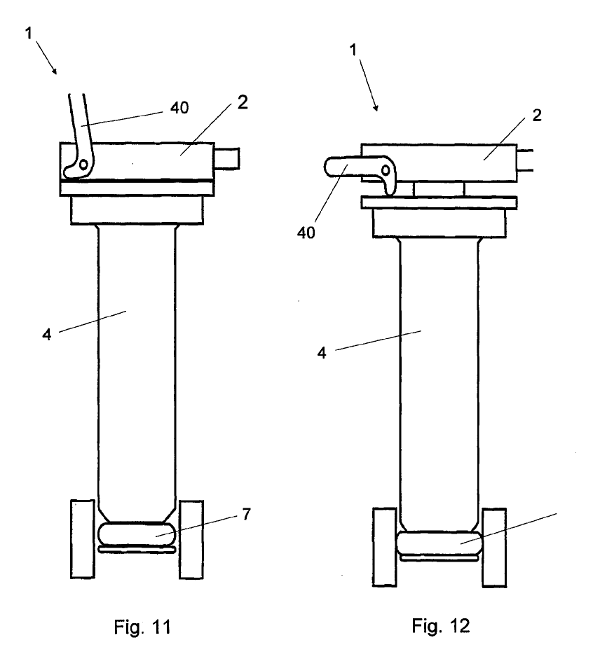

FIGS. 11 and 12 show an alternative embodiment of the tensioning and/or fastening element, and

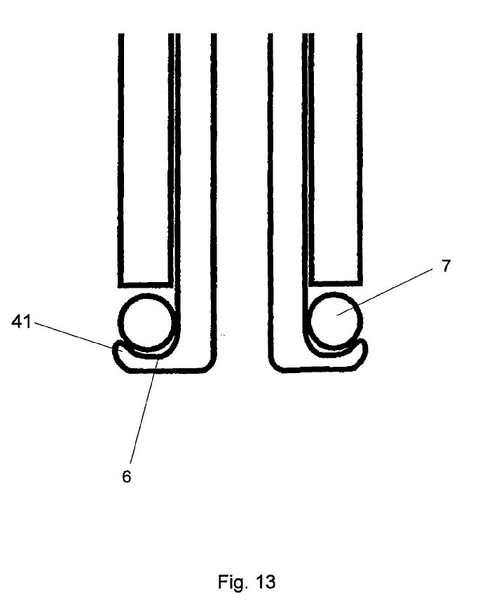

FIG. 13 shows a detailed view of an alternative embodiment of a surgical instrument according to the invention.

In FIG. 1, 1 denotes a surgical instrument for introducing a freely-flowing medium for lifting the Schneiderian membrane from the jaw bone when performing a percrestal sinus lift. The instrument consists of a body 2, wherein a sleeve 4 extends around the elongate body 2 of the surgical instrument 1. An annular face 6 is arranged at the distal end 5 of the body 2, with a sealing element or an annular seal 7 coming to rest on said annular face. Arranged within the interior of the body 2 there is a channel 9 for the supply of freely-flowing medium supplied via the connection 10. 11 denotes a fixing screw 11 that interacts with the thread 12. The penetration depth of the surgical instrument into the bore channel 3 in the jaw bone 13 is restricted by stops 14 and 15, wherein, during use, stop 14 comes to rest on the gum and stop 15 becomes effective if implantation rails are used during the operation and the stops are in both cases finely tuned with cylindrical disks to the planned depth.

FIG. 2 illustrates the surgical instrument as per FIG. 1, wherein in this case the displaceable sleeve was displaced in the direction of the arrow 16 relative to the body 2 and so the annular seal 7, which comes to rest on the annular face 6, was compressed and deformed radially outward such that the annular seal 7 comes to rest on the cut surface 17 of the jaw bone 13. As shown in FIG. 2, this advanced position of the sleeve 4 can be fixed by means of the screw 11, which was advanced on the thread 12 by screwing.

FIG. 3 shows an alternative embodiment of a surgical instrument according to the invention, wherein the same parts are provided with the same reference signs. In this embodiment, it is merely the annular seal 7 that has an alternative design. As per the embodiment according to FIG. 3, the annular seal 7 is not designed as an O-ring seal, but as a profiled joint, which in turn can be compressed and deformed radially outward by displacing the sleeve 4.

FIG. 4 in turn illustrates the surgical instrument as per FIG. 3 in a tensioned state, wherein the sleeve 4 is displaced in the direction of the arrow 16 relative to the body 2, the annular seal or the sealing element 7 is deformed radially outward and comes to rest on the cut surfaces 17 of the jaw bone 13.

FIG. 5 shows an alternative embodiment of the surgical instrument according to the invention, in which the sealing element is designed in the form of an inflatable balloon. The sleeve can be dispensed with in this embodiment, wherein, in this case, a channel 18 is arranged in the body 2 of the surgical instrument, which channel can via a connection 19 be fed with a medium that can fill a cavity 20 formed by a recess in the body 2 of the surgical instrument and an envelope 21.

FIG. 6 shows the surgical instrument as per FIG. 5 in a state fixed in the bore channel, wherein it can be seen that the introduction of a medium into the connection 19 results in the medium inflating the envelope 21 like a balloon through the channel 18 into the cavity 20 and said envelope coming to rest on the cut surface 17 of the jaw bone 13. In this state, the free-flowing medium for lifting the Schneiderian membrane from the jaw bone 13 in turn can be introduced via the connection 10 and the channel 9.

FIGS. 7 and 8 now illustrate an alternative embodiment, in which, instead of a screw, provision is made for a slider 22 for affixing the sleeve 4 in the tensioned state, which slider has a face 23 arranged at an angle to the axis of the surgical instrument 1, which face interacts with a corresponding face 24 on the sleeve 4 of the surgical instrument 1 and so sliding the slider in the direction of the arrow 25 results in a displacement of the sleeve 4 in the direction of the arrow 26, as a result of which the annular seal 10 is in a relaxed state. When displacing the slider 22 in the direction of the arrow 27 in FIG. 8, the sleeve 4 is displaced in the direction of the arrow 28 relative to the body 2, as a result of which the annular seal 10 is compressed and deformed radially outward.

FIG. 9 now illustrates the drill set to be used when performing a percrestal sinus lift, wherein it can be seen that a pilot bore is drilled by means of the drill of the first type 29 up to almost the sinus-side end 30 of the jaw bone 13. Using a first drill of the second type 31, the cylindrical body 32 of which has a non-cutting design and which thus comes to rest on the base of the bore 33, it is now possible to drill up to the vicinity of the Schneiderian membrane 35, without the risk of perforating the latter, by using the drill pin 34, with the penetration of the membrane 35 already being possible in this step if the drill pin 34 has a sufficient length. However, if need be, the jaw bone 13 can be penetrated by means of a further drill of the second type 36, the drill pin 37 of which has a greater length.

FIG. 10 shows that there is conventional cooling and rinsing at the tip of the drill pin 37 using, for example, Ringer's solution 38 and so a swimming screen 39 is formed when the sinus-side edge 30 of the jaw bone 13 is penetrated, which screen protects the Schneiderian membrane 35 from the rotating tip of the drill pin 37.

FIGS. 11 and 12 show the function of an eccentric lever as a tensioning and/or fastening element. The eccentric lever 40 is attached to the body 2 of the surgical instrument 1 in a rotatable fashion. When, as shown in FIG. 12, the lever is tilted, the front region of the eccentric lever 40 pushes on the upper edge of the sleeve 4 and so the latter is displaced distally and relative to the body. This leads to the desired deformation of the sealing element 7.

FIG. 13 shows a detail of an alternative embodiment of the surgical instrument 1, in which the annular face 6 has means for securing the sealing element 7 against slipping off. Here, arranged in the radially outer region of the annular face 6 is an encircling elevation 41, which securely holds the sealing element on the annular face.

1. A method for performing a percrestal sinus lift comprising:
creating a bore channel via drilling, milling and/or hammering methods,
lifting a Schneiderian membrane from a jaw bone using a free-flowing medium supplied through an opened-up bore channel, and introducing bone-replacement material, wherein a supply line is inserted into the bore channel prior to supplying the free-flowing medium and is detachably affixed in a sealing fashion in the bore channel or to an edge thereof, and wherein the free-flowing medium is introduced through the supply line.

2. The method as claimed in claim 1, wherein the supply line is affixed in the bore channel or to the edge thereof via a flexible annular seal.

3. The method as claimed in claim 2, wherein the annular seal is deformed or expanded in radial direction and pressed against an inner wall of the bore channel.

4. The method as claimed in claim 1, wherein an annular seal designed as a tubular seal or balloon seal is pressed against an inner wall of the bore channel by application of pressure.

5. The method of claim 1, wherein the supply line is affixed in the bore channel in a sealing fashion at its front end in insertion direction.

6. The method as claimed in claim 1, wherein a gel is used as the free-flowing medium.

7. The method as claimed in claim 1, wherein a highly viscous bone-replacement material is the free-flowing medium.

8. A surgical instrument for introducing a free-flowing medium for lifting a Schneiderian membrane from a jaw bone when performing a percrestal sinus lift comprising a supply line, which can be inserted into the bore channel and has at least one sealing element for affixing the supply line in a bore channel or to an edge thereof in a sealing and detachable fashion, wherein a sleeve that can be displaced relative to the supply line in an axial direction is guided along the supply line and wherein the sealing element is arranged between an end face of the sleeve and a face, more particularly an annular face, projecting from the supply line in radial direction, can be deformed by axial pressing of the sleeve in the radial direction, and is designed such that it can be pressed against an inner wall of the bore channel.

9. The surgical instrument as claimed in claim 8, wherein the sealing element has at least one flexible annular seal.

10. The surgical instrument as claimed in claim 8, wherein the annular seal is designed such that it can be deformed in the radial direction and can be pressed against the inner wall of the bore channel.

11. The surgical instrument as claimed in claim 8, wherein the annular face has securing means to prevent the sealing element from slipping off.

12. The surgical instrument as claimed in claim 8, wherein the end face of the sleeve facing the sealing element is designed as an angled face, more particularly a frustum-shaped face, facing away from the supply line.

13. The surgical instrument as claimed in claim 8, wherein the sleeve interacts with a tensioning and/or fastening element for tensioning or fastening the sleeve in a position pressed against the sealing element.

14. The surgical instrument as claimed in claim 13, wherein the tensioning and/or fastening element is formed by a screw that can be screwed against the sleeve in axial direction.

15. The surgical instrument as claimed in claim 13, wherein the tensioning or fastening element is formed by a slider acting like a wedge, the face of the slider angled relative to the axis of the sleeve interacts with a ramp face of the sleeve during the lateral insertion in order to press the sleeve against the sealing element in axial direction.

16. The surgical instrument as claimed in claim 8, wherein the sealing element is arranged at a front end of the supply line in insertion direction.

17. The surgical instrument as claimed in claim 8, wherein the supply line and/or the sleeve have/has a stop for limiting insertion depth into the bore channel.

18. An instrument set for performing a percrestal sinus lift, comprising a drilling, milling and/or hammering instrument for creating a bore channel, an instrument as claimed in claim 8 for supplying a free-flowing medium via the bore channel in order to lift the Schneiderian membrane from the jaw bone and an instrument, preferably separate from the other instruments, for introducing a bone-replacement material.

19. A drill set for use when performing a percrestal sinus lift, wherein at least one drill of a first type, which is designed as a pilot drill, and at least a first drill of a second type, which has a non-cutting cylindrical body with a diameter not exceeding the drill diameter of the drill of the first type and a drill pin protruding from the distal end face of the cylindrical body by a defined length.

20. The drill set as claimed in claim 19, wherein at least a second drill of the second type has a drill pin protruding from the distal end face of the cylindrical body by a greater defined length than that length of the drill pin of the first drill of the second type.

21. The drill set as claimed in claim 19, wherein the drills of the first and the second type each have channels for supplying and/or discharging cooling and/or rinsing liquid, wherein the channels for supplying the cooling and/or rinsing liquid preferably open into a region of the drill pin.

22. The drill set as claimed in claim 19, wherein the drill pin of the at least one drill of the second type is designed as a gun drill with a semicircular drill tip.

23. The drill set as claimed in claim 19, wherein the drill pin of the at least one drill of the second type (31) is designed as a diamond-finished drill tip.
